(19) United States
(12) Patent Application Publication
(10) Pub. No.: US 2021/0395690 A1
(43) Pub. Date: Dec. 23, 2021

(54) ANCHORAGE-INDEPENDENT CELLS AND USE THEREOF

(71) Applicant: Yissum Research Development Company of the Hebrew University of Jerusalem Ltd., Jerusalem (IL)

(72) Inventors: Yaakov NAHMIAS, Mevaseret Zion (IL); Merav Cohen, Jerusalem (IL)

(73) Assignee: Yissum Research Development Company of the Hebrew University of Jerusalem Ltd., Jerusalem (IL)

(21) Appl. No.: 17/292,303

(22) PCT Filed: Nov. 7, 2019
(86) PCT No.: PCT/IL2019/051219

§ 371 Date: May 7, 2021

Related U.S. Application Data
(60) Provisional application No. 62/757,275, filed on Nov. 8, 2018.

(51) Int. Cl. C12N 5/077

(52) U.S. Cl. CPC C12N 5/0656; C12N 2527/00; C12N 5/0653

(57) ABSTRACT

An enriched population of connective tissue cells that are capable of anchorage-independent growth are provided. Compositions comprising those cells, as well as methods of producing those cells are also provided.

### CROSS REFERENCE TO RELATED APPLICATIONS

This application claims priority from U.S. Patent Application No. 62/757,275, filed on Nov. 8, 2018, entitled “ANCHORAGE-INDEPENDENT CELLS AND USE THEREOF”, the contents of all of which are incorporated by reference herein in their entirety.

### FIELD OF INVENTION

The present invention is in the field of generation of anchorage-independent cells from anchorage-dependent ones.

### BACKGROUND OF THE INVENTION

Tissue culture of both immortalized and primary cells can be done with both adherent and suspension cells. Growing cells in suspension has the obvious advantage of being able to culture a far greater density of cells in one vessel. Bioreactors, and other suspension reactors allow for densities of tens of millions of cells per mL to be cultured. However, many cell types can only be cultured as adherent cells and thus the number of cells that can be practically cultured at once is severely limited. For commercial cultures where maximizing cell number is paramount (such as for vaccine production) converting an adherent cell line to one that can be grown in suspension is of great interest. Grown cells in suspension as single cells, without aggregates or microcarriers, is even more ideal.

Up to this point, very few cell lines can be easily converted from adherent to suspension. Mesenchymal stem cells, which grow adherently, can also be grown in suspension as spheroids. However, MSCs do not grow as single cell cultures, thus they still require anchorage to other cells when in suspension. Amniocytes, retinal cells, and embryonic stem cells have been successfully cultured in suspension, however, fibroblasts have yet to be practically converted to suspension cells that grow without any contact (cell contact or microcarriers). Fibroblasts have been found to be particularly difficult to culture in suspension (Jordan et al., An avian cell line designed for production of highly attenuated viruses, Vaccine 2009). Fibroblasts have been grown with carriers such as methyl cellulose, and some culture conditions have generated aggregates and/or unhealthy cells, however, a fully suspended and healthy fibroblast cell line is still greatly in need.

### SUMMARY OF THE INVENTION

The present invention provides an enriched population of connective tissue cells that are capable of anchorage-independent growth. Compositions comprising those cells, as well as methods of producing those cells are also provided.

According to a first aspect, there is provided an enriched population of connective tissue cells, wherein at least 70% of the connective tissue cells are capable of anchorage-independent growth.

According to another aspect, there is provided a composition comprising an enriched population of the invention.

According to another aspect, there is provided a method of producing an anchorage-independent cell line, the method comprising, growing aggregates of an anchorage-dependent cell line in vitro, mechanically disrupting the aggregates into single cells in liquid, and growing the single cells in a liquid culture for at least 4 generations; thereby producing the anchorage-independent cell line.

A method for decreasing the doubling time of an anchorage-dependent cell line, the method comprising, growing aggregates of the anchorage-dependent cell line in vitro, mechanically disrupting the aggregates into single cells in liquid, and growing the single cells in a liquid culture for at least 4 generations; thereby increasing the doubling time of the anchorage-dependent cell line.

According to some embodiments, at least 95% of the connective tissue cells are capable of anchorage-independent growth. According to some embodiments, 100% of the connective tissue cells are capable of anchorage-independent growth.

According to some embodiments, at least 20% of the anchorage independent connective tissue cells are actively proliferating.

According to some embodiments, the anchorage-independent connective tissue cells are capable of anchorage-independent growth for at least 4 cellular divisions.

According to some embodiments, the connective tissue cells are fibroblasts or a cell type that can naturally be differentiated from a fibroblast. According to some embodiments, the connective tissue cells are fibroblasts. According to some embodiments, the cell type that can naturally be differentiated from a fibroblast is selected from the group consisting of: a chondrocyte, an adipocyte, an osteoblast, an osteocyte, a myofibroblast, a myoblast and a myocyte.

According to some embodiments, the anchorage-independent connective tissue cells comprise an intact plasma membrane.

According to some embodiments, the enriched population comprises a doubling time of 50 hours or less. According to some embodiments, the enriched population comprises a doubling time of between 18 and 22 hours.

According to some embodiments, the anchorage-independent connective tissue cells grow in liquid culture as at least 85% single cells.

According to some embodiments, the connective tissue cells are mammalian connective tissue cells. According to some embodiments, the connective tissue cells are avian connective tissue cells.

According to some embodiments, the connective tissue cells are capable of producing cultured meat.

According to some embodiments, a yield of virus produced by the enriched population after infection is equal to or greater than a yield produced by an equal number of anchorage-dependent connective tissue cells after infection.

According to some embodiments, the anchorage-independent fibroblasts are incapable of adherent growth.

According to some embodiments, a composition of the invention further comprises a liquid in vitro cellular growth medium, wherein at least 70% of the anchorage-independent connective tissue cells are not adhered to a surface.

According to some embodiments, the in vitro cellular growth medium is devoid of serum.

According to some embodiments, the anchorage-independent connective tissue cells are at a density of greater than 5 million cells/mL of in vitro cellular growth medium.

According to some embodiments, a composition of the invention is devoid of microcarrier beads.

According to some embodiments, a composition of the invention further comprises a matrix. According to some embodiments, the matrix is a vegetable-derived matrix, wherein the anchorage-independent connective tissue cells are differentiated into adipocytes and wherein the composition is cultured meat. According to some embodiments, the matrix is selected from a collagen matrix, a dermal matrix and a substitute dermal matrix and wherein the composition is leather.

According to some embodiments, the growing aggregates is performed in a non-adherent dish.

According to some embodiments, the growing single cells is performed in shaker or spinner flasks.

According to some embodiments, the growing in shaker or spinner flasks comprises at most one passage at spin speeds below 40 RPM, followed by at least 3 passages at spin speeds of between 80 and 100 RPM.

According to some embodiments, the anchorage-dependent cell line is a fibroblast cell line, and wherein the anchorage-independent cell line is a fibroblast cell line.

According to some embodiments, the anchorage-independent cell line is an enriched population of the invention.

According to some embodiments, the decreasing lowers the doubling time to 50 hours or less.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: A photograph of DF-1 fibroblasts grown on plastic.

FIG. 2: A photograph of Spheroids of DF-1 fibroblasts forming on Aggrewell, 16 hours post seeding.

FIG. 3: Photographs of Spheroids, large aggregates, and single cells growing on non-adherent 10-cm petri dishes (day 4).

FIG. 4: A bar graph of doubling time of DF-1 cells in shaker flasks at various passages.

FIG. 5: A photograph of DF-1 anchorage-independent cells at passage 34 growing as a single cell suspension.

FIGS. 6A-E: (6A-D) Combine bar and line graphs of doubling time and viability of (6A) FMT-SCF-1, FMT-SCF-2, FMT-SCF-3, (6B) FMT-SCF-4, FMT-SCF-5, (6C) FMT-SBF-1, FMT-SCF-2, and (6D) FMT-SCF-3 by passage number. Bars represent the doubling time at each passage, and the line represents viability. (6E) Micrographs of cellular suspensions of the various cell lines showing predominantly (>90%) growth as single cells.

FIGS. 7A-B: Micrographs of (7A) chicken and (7B) bovine adipocytes stained with LipidTOX at day 4 and day 7 from the start of the adipocyte culture.

FIGS. 8A-F: Photographs of (8A-C) cultured chicken nuggets and (8D-F) cultured beef (8A) Photograph of grilled cultured chicken teriyaki nuggets. (8B) Photograph of a cultured chicken nugget (bottom left) and a farm grown chicken nugget (top right). (8C) Photograph of a cross-section of cultured (bottom left) and farm grown (top right) chicken nuggets. (8D-F) Photographs of (8D) uncooked cultured beef in a bowl, (8E) cultured beef kabobs and (8F) a cross-section of a cultured beef kabob.

### DETAILED DESCRIPTION OF THE INVENTION

The present invention, in some embodiments, provides an enriched population of connective tissue cells that are capable of anchorage-independent growth. The present invention further concerns compositions comprising those cells, and a method of producing those cells. A method of decreasing the doubling time of an anchorage-dependent cell line is also provided.

By a first aspect, there is provided an enriched population of connective tissue cells, wherein said enriched population comprises connective tissue cells capable of anchorage-independent growth.

By another aspect, there is provided a population of anchorage-independent connective tissue cells.

As used herein, the term “anchorage-independent growth” refers to cellular growth while not adhered to a substrate. Anchorage-independent growth may also be referred to as non-adherent growth, or liquid culture. Many cell lines require a substrate on which to adhere in order to growth. Similarly, many cells in an organism require cell-cell contact in order to grow. In some embodiments, anchorage-independent growth is growth wherein the cell is surrounded by media. In some embodiments, anchorage-independent growth is wherein a cell is not contacting another cell or surface. In some embodiments, the surface is an artificial surface such as a tissue culture dish, or a microbead. In some embodiments, the surface is another cell. In some embodiments, anchorage-independent growth is not growth as a spheroid or aggregate. In some embodiments, anchorage-independent growth is growth as single cells in solution. As used herein, the terms “connective tissue cells capable of anchorage-independent growth” and “anchorage-independent connective tissue cells” are synonymous and used interchangeably.

In some embodiments, at least 50%, 55%, 60%, 65%, 70%, 75%, 80%, 85%, 90%, 95%, 97%, 99% or 100% of the anchorage-independent connective tissue cells grow in liquid culture as single cells. Each possibility represents a separate embodiment of the invention. In some embodiments, at least 70% grow as single cells. In some embodiments, at least 75% grow as single cells. In some embodiments, at least 80% grow as single cells. In some embodiments, at least 85% grow as single cells. In some embodiments, at least 90% grow as single cells. In some embodiments, at least 95% grow as single cells. In some embodiments, at least 97% grow as single cells. In some embodiments, at least 99% grow as single cells. In some embodiments, between 70 and 90% of anchorage-independent connective tissue cells grow in liquid culture as single cells. In some embodiments, at least 90% grow as single cells. In some embodiments, between 70 and 80% of anchorage-independent connective tissue cells grow in liquid culture as single cells. In some embodiments, the liquid culture comprises serum. In some embodiments, the liquid culture is serum-free. In some embodiments, the liquid culture comprises serum and at least 90% of cells grow as single cells. In some embodiments, the liquid culture is serum-free and between 70 and 80% of cells grow as single cells.

As used herein, the term “connective tissue cells” refers to the various cell types that make up connective tissue. In some embodiments, connective tissue cells are selected from fibroblasts, cartilage cells, bone cells, fat cells and smooth muscle cells. In some embodiments, connective tissue cells are selected from the group consisting of chondrocytes, adipocytes, osteoblasts, osteocytes, myofibroblasts, satellite cells, myoblasts and myocytes. In some embodiments, connective tissue cells are selected from the group consisting of, adipocytes, osteoblasts, osteocytes, myofibroblasts, satellite cells, myoblasts and myocytes. In some embodiments, connective tissue cells are fibroblasts. In some embodiments, the fibroblasts are not embryonic fibroblasts. In some embodiments, the fibroblasts are embryonic fibroblasts. In some embodiments, the fibroblasts are fetal fibroblasts. In some embodiments, the fibroblasts are dermal fibroblasts. In some embodiments, connective tissue cells are fibroblasts or a cell type that can be differentiated from a fibroblast. In some embodiments, connective tissue cells are not mesenchymal stem cells (MSCs). In some embodiments, connective tissue cells are not cells derived from MSCs. In some embodiments, connective tissue cells are cell that cannot be derived from MSCs. In some embodiments, the cell type can be naturally differentiated form a fibroblast. In some embodiments, the cell type results from natural fibroblast differentiation. As used herein, the “term natural differentiation” is used to refer to a differentiation that occurs in nature and not a trans-differentiation such as can artificially be achieved in a laboratory. In some embodiments, the natural differentiation is not de-differentiation. In some embodiments, a cell type that can naturally be differentiated form a fibroblast is selected from the group consisting of: a chondrocyte, an adipocyte, an osteoblast, an osteocyte, a myofibroblast, a myoblast and a myocyte. In some embodiments, a cell type that can naturally be differentiated form a fibroblast is selected from the group consisting of: an adipocyte, an osteoblast, an osteocyte, a myofibroblast, a myoblast and a myocyte. In some embodiments, a cell type that can naturally be differentiated form a fibroblast is an adipocyte. In some embodiments, the connective tissue cell is not a pluripotent cell. In some embodiments, the connective tissue cell is not a mesenchymal stem cell.

In some embodiments, the connective tissue cells are mammalian cells. In some embodiments, the mammal is a bovine. In some embodiments, the bovine is a cow. In some embodiments, the connective tissue cells are avian cells. In some embodiments, the connective tissue cells are fish cells. In some embodiments, the connective tissue cells are from an edible animal. In some embodiments, the cells are from livestock animals. In some embodiments, a livestock animal is selected from a cow, a pig, a goat, a sheep, a chicken, a fish and a turkey. In some embodiments, a livestock animal is selected from a cow, a pig, a goat, a sheep, a chicken, a fish, a duck, a goose and a turkey. In some embodiments, a livestock animal is selected from a cow, a pig, a goat, a sheep, a chicken, a duck, a goose and a turkey. In some embodiments, the connective tissue cells are selected from avian cells and bovine cells. In some embodiments, the bovine cells are cow cells. In some embodiments, the avian cells are chicken cells. In some embodiments, the connective tissue cells are selected from cow cells and chicken cells. In some embodiments, the chicken cells are chicken fibroblasts. In some embodiments, the cow cells are cow fibroblasts. In some embodiments, the chicken fibroblasts are DF-1 cells. In some embodiments, the cells are immortalized. In some embodiments, the cells are not immortalized. In some embodiments, the cells are derived from primary cells.

In some embodiments, at least 50%, 55%, 60%, 65%, 70%, 75%, 80%, 85%, 90%, 95%, 97%, 99% or 100% of the enriched population is connective tissue cells capable of anchorage-independent growth. Each possibility represents a separate embodiment of the invention. In some embodiments, 100% of the enriched population is connective tissue cells capable of anchorage-independent growth. In some embodiments, the enriched population is a population of connective tissue cells capable of anchorage-independent growth. In some embodiments, the enriched population is a population of anchorage-independent connective tissue cells. In some embodiments, the population of anchorage-independent connective tissue cells is essentially pure. In some embodiments, the population of anchorage-independent connective tissue cells is devoid of anchorage-dependent cells. In some embodiments, essentially pure comprises at least 70, 75, 8, 85, 90, 95, 97, 99 or 100% purity. Each possibility represents a separate embodiment of the invention.

In some embodiments, at least 50%, 55%, 60%, 65%, 70%, 75%, 80%, 85%, 90%, 95%, 97%, 99% or 100% of the connective tissue cells are capable of anchorage-independent growth. Each possibility represents a separate embodiment of the invention. In some embodiments, at least 50%, 55%, 60%, 65%, 70%, 75%, 80%, 85%, 90%, 95%, 97%, 99% or 100% of the connective tissue cells are anchorage-independent cells. Each possibility represents a separate embodiment of the invention. In some embodiments, at least 70% of the cells are capable of anchorage-independent growth. In some embodiments, at least 95% of the cells are capable of anchorage-independent growth. In some embodiments, at least 99% of the cells are capable of anchorage-independent growth. In some embodiments, at least 100% of the cells are capable of anchorage-independent growth. In some embodiments, the enriched population does not comprise cells growing adherently. In some embodiments, the enriched population does not comprise adherent cells.

In some embodiments, at least 20%, 25%, 30%, 35%, 40%, 45%, 50%, 55%, 60%, 65%, 70%, 75%, 80%, 85%, 90%, 95%, 97%, 99% or 100% of the anchorage independent connective tissue cell are actively proliferating. Each possibility represents a separate embodiment of the invention. Active proliferation can be assessed by Ki67 staining, in which proliferative cells stain positive. In some embodiments, the cells are not mutagenized. In some embodiments, the cells are not irradiated.

In some embodiments, the cells capable of anchorage-independent growth are alive during growth in medium and/or on non-adherent plates. In some embodiments, the live cells have an intact plasma membrane. Live/dead staining with a live/dead dye such as PI, Hoechst and Trypan Blue can be performed to assess the percentage of live cells as well as assessing plasma membrane integrity. In some embodiments, the enriched population comprises at least 50%, 55%, 60%, 65%, 70%, 75%, 80%, 85%, 90%, 95%, 97%, 99% or 100% live cells. Each possibility represents a separate embodiment of the invention.

In some embodiments, the anchorage-independent cells are capable of anchorage-independent growth for at least 1, 2, 3, 4, 5, 7, 10, 12, 15, 17, 20, 22, 25, 27, 30, 32, 34, 35, 37 or 40 cellular divisions. Each possibility represents a separate embodiment of the invention. A cellular division is also referred to herein as a passage. In some embodiments, the anchorage-independent cells are capable of anchorage-independent growth indefinitely. In some embodiments, the anchorage-independent cells are capable of anchorage-independent growth for at least 1 passage. anchorage-independent cells are capable of anchorage-independent growth for at least 4 passages. anchorage-independent cells are capable of anchorage-independent growth for at least 34 passages. In some embodiments, the anchorage-independent cells are incapable on adherent growth. In some embodiments, at least 50%, 55%, 60%, 65%, 70%, 75%, 80%, 85%, 90%, 95%, 97%, 99% or 100% of the anchorage-independent cells are incapable on adherent growth. Each possibility represents a separate embodiment of the invention.

In some embodiments, the enriched population comprises a doubling time of less than 60, 55, 50, 45, 40, 39, 39, 37, 36, 35, 34, 33, 32, 31, 30, 29, 28, 27, 26, 25, 24, 23, 22, 21 or 20 hours. Each possibility represents a separate embodiment of the invention. In some embodiments, doubling time is average doubling time. In some embodiments, the doubling time is 50 hours of less. In some embodiments, the doubling time is 40 hours of less. In some embodiments, the doubling time is 35 hours of less. In some embodiments, the doubling time is 30 hours of less. In some embodiments, the doubling time is 28 hours or less. In some embodiments, the doubling time is 26 hours or less. In some embodiments, the doubling time is 25 hours or less. In some embodiments, the doubling time is 22 hours or less. In some embodiments, the enriched population comprises a doubling time of between 22 and 18 hours. In some embodiments, the enriched population comprises a doubling time of between 25 and 18 hours. In some embodiments, the enriched population comprises a doubling time of between 21 and 26 hours. In some embodiments, the enriched population comprises a doubling time of between 22 and 26 hours. In some embodiments, the enriched population comprises a doubling time of between 21 and 27 hours. In some embodiments, the enriched population comprises a doubling time of between 26 and 34 hours. In some embodiments, the enriched population comprises a doubling time of between 28 and 32 hours.

In some embodiments, the doubling time is about the same as a doubling time of an anchorage-dependent cell line. In some embodiments, the doubling time is about the same as a doubling time of an equivalent anchorage-dependent cell or cell line. In some embodiments, the enriched population comprises a decreased doubling time as compared to an anchorage-dependent cell line of the same cell type. In some embodiments, the enriched population comprises a decreased doubling time as compared to a suspension cell line. In some embodiments, the enriched population comprises a decreased doubling time as compared to embryonic stem cells (ESCs). The doubling time of ESCs is well known in the art and is between 36-40 hours and averages about 38 hours.

In some embodiments, the decrease is at least a 10%, 15%, 20%, 25%, 30%, 35%, 40%, 45%, 50%, 55%, 60%, 65%, 70%, 75%, or 80% decrease in doubling time. Each possibility represents a separate embodiment of the invention. In some embodiments, the anchorage-independent DF-1 cell line has a doubling time of between 13-24 hours. In some embodiments, the anchorage-dependent DF-1 cell line has a doubling time of between 13-24 hours. In some embodiments, suspension cell lines have a doubling time of 24-60 hours.

In some embodiments, the enriched population of connective tissue cells expresses cellular markers of that connective tissue. In some embodiments, the cellular markers are expressed at levels comparable to the levels expressed in anchorage-dependent cells of the same connective tissue. In some embodiments, comparable is within +/−5%, 10%, 15%, 20%, 25%, 30%, 35%, 40%, 45% or 50% of the levels in the anchorage-dependent cells. Each possibility represents a separate embodiment of the invention. In some embodiments, at least 1, 2, 3, 4, or 5 cellular markers of the cell type are expressed. Each possibility represents a separate embodiment of the invention. In some embodiments, anchorage-independent cells are still identifiable as of the connective tissue cell type by expression of the markers. In some embodiments, the anchorage-independent connective tissue cells express cellular marker of the equivalent anchorage-dependent cells. In some embodiments, the markers are expressed at comparable levels.

As used herein, the term “equivalent anchorage-dependent cells” refers to the anchorage-dependent cells, who, by the methods of the invention have been converted into anchorage-independent cells. The cells are equivalent as they are the same cell type and have not been modified other than the ability to grow non-adherently has been altered.

Methods of measuring gene and protein expression are well known in the art. The cellular markers for a particular cell type may be protein markers, and/or RNA markers. For example, RNA may be measured by RT-PCR, quantitative PCR, northern blotting or in situ hybridization to name but a few methods. Protein expression may be measured by FACS, western blotting, ELISA or immunohistochemistry/immunostaining for example. Any method that can accurately measure expression of the cellular markers may be employed.

Markers of various connective tissue cell types are well known in the art, and include, for example, CD34, alpha-actin, and fibroblast-specific protein 1 (FSP1) as markers of fibroblasts; aggrecan, collagen type II, and CRTAC1 for chondrocytes; Pref-1, FABP4, adiponectin and leptin for adipocytes; DMP-1, FGF-23 and biglycan for osteocytes, alkaline phosphatase, BAP1, collagen I and osteocalcin for osteoblasts, and alpha-smooth muscle actin, calponin 1, VE-cadherin and desmin in smooth muscle myoblasts. Other examples of markers can be found on the websites of many companies that produce antibodies, such as R&D Systems (rndsystems.com), and Cell Signaling Technology (cellsignal.com) to name but a few.

In some embodiments, the connective tissue cells are capable of producing cultured meat. In some embodiments, the connective tissue cells are for use in producing cultured meat.

By another aspect, there is provided a use of a population of the invention for producing cultured meat.

By another aspect, there is provided a composition comprising a population of the invention.

In some embodiments, the composition further comprises a matrix. In some embodiments, the matrix is an organic matrix. In some embodiments, the matrix is an inorganic matrix. In some embodiments, the matrix is a collagen or collagen-based matrix. In some embodiments, the matrix is a dermal matrix. In some embodiments, the matrix is a dermal substitute matrix. In some embodiments, the matrix is a serum-free matrix. In some embodiments, the matrix is a scaffold. In some embodiments, the scaffold is a porous scaffold. Examples of porous scaffolds include, but are not limited to polylactic acid, polyglycolic acid, poly(lactic-glycolic acid, PLGA, and hydroxypropyl cellulose scaffolds. In some embodiments, the matrix is biodegradable.

In some embodiments, the matrix is a plant-derived matrix. In some embodiments, the matrix is a vegetable-derived matrix. In some embodiments, the plant is a vegetable. In some embodiments, the plant is selected from cereal, gluten and legume. In some embodiments, the plant is selected from the legumes, the Fabaceae family, the cereal family, and the pseudocereal family. The Fabaceae family includes, for example, alfalfa, peas, beans, lentils, carob, soybeans, and peanuts. The cereal family includes, for example, maize, rice, wheat, barley, sorghum, millet, oats, rye, tritcale, and fonio. The pseudocereal family includes, for example, buckwheat, quinoa and chia. In some embodiments, the legume is so or pea. In some embodiments, the legume is soy. In some embodiments, the plant-derived matrix is a soy-protein matrix. In some embodiments, the plant-derived matrix is a pea-protein matrix.

In some embodiments, the cells of the invention are cultured in the matrix. In some embodiments, the cells of the invention are layered on the matrix. In some embodiments, the cells of the invention are mixed with the matrix. In some embodiments, the cells of the invention and a plant protein are mixed. In some embodiments, the plant protein is selected from pea protein and soy protein. In some embodiments, the plant protein is soy protein. In some embodiments, the protein is a high-moisture extrusion of the protein.

In some embodiments, the matrix is in a perfusion system. In some embodiments, the matrix is an edible hollow fiber cartridge. In some embodiments, the matrix further comprises a nutrient supply homogenously distributed throughout the matrix. In some embodiments, the matrix further comprises an integrated vascular network. For example, the fibers of the cartridge are made from edible natural or synthetic polymers, such as cellulose (FiberCell, #C3008), cellulose acetate and the cells form a mass surrounding the fibers. Cellulose is FDA approved as GRAS, and used to control moisture and stabilizer shredded cheese, bread, and various sauces.

In some embodiments, the composition comprising the cells of the invention and the matrix is cultured meat. In some embodiments, the cells in the cultured meat are fibroblasts. In some embodiments, the cells in the cultured meat are adipocytes. In some embodiments, the cells in the cultured meat are myoblasts. In some embodiments, the cultured meat is edible meat. According to some embodiments of the invention, the edible meat is in a form of a patty of nugget with a density in the range of about 100×10\hat{ }6 cells/gram to about 500×10\hat{ }6 cells/gram, e.g., about 200×10\hat{ }6 cells/gram.

In some embodiments, the cultured meat comprises at least 5, 10, 15, 20, 25, 30, 35, 40, 45 or 50% cells. Each possibility represents a separate embodiment of the invention. In some embodiments, the cultured meat comprises at least 20% cells. In some embodiments, the cultured meat comprises at least 30% cells. In some embodiments, the cultured meat is cultured chicken and comprises at least 20% chicken adipocytes. In some embodiments, the cultured meat is cultured beef and comprises at least 30% beef adipocytes. In some embodiments, the percentage is percentage of weight. In some embodiments, the percentage is percentage of mass. In some embodiments, the percentage is percentage of volume.

In some embodiments, the composition comprising the cells of the invention and the matrix is leather. In some embodiments, the leather is faux-leather. In some embodiments, a composition comprising cells of the invention and a collagen matrix, a dermal matrix or a substitute dermal matrix is leather. In some embodiments, the composition is configured as leather. In some embodiments, the composition is configured to look and/or feel like leather. In some embodiments, the cells in the leather are fibroblasts.

As used herein, the term “cultured meat” refers to meat produced by in vitro cultivation of animal cells. In some embodiments, the enriched population is grown without serum for the production of cultured meat. In some embodiments, the enriched population for use in producing cultured meat is not genetically modified. In some embodiments, the enriched population is differentiated to a particular cell type for the production of culture meat. In some embodiments, the particular cell type is selected from adipocytes, myocytes, osteoblasts, osteocytes and chondrocytes. In some embodiments, the particular cell type is selected from adipocytes, myocytes, osteoblasts, and osteocytes. In some embodiments, the enriched population is differentiated to a particular tissue for the production of cultured meat. In some embodiments, the particular tissue is selected from fat, muscle, bone and cartilage.

Methods of producing cultured meat are well known in the art and any known method may be employed. One such method is found in International Patent Application PCT/IL2017/050790 which is herein incorporated by reference in its entirety.

In some embodiments, the enriched population is for use in producing a product of interest. In some embodiments, the product of interest is a vaccine. In some embodiments, the product of interest is a glycosylated protein. In some embodiments, the product of interest is a virus or viral fragment. In some embodiments, the virus is selected from a live virus, a mutated virus, an attenuated virus and a viral fragment. would be commercially interesting to produce.

By another aspect, there is provided a use of the enriched population of the invention in producing a vaccine. Vaccine producing in cultured fibroblasts is well known in the art, and may include infecting the fibroblasts with a live, and/or attenuated virus such that the virus will increase within the cells to yield a large amount of virus (in the supernatant or from lysed cells) that may be used as a vaccine or a component in a vaccine.

The term “virus” as used herein includes not only naturally occurring viruses but also attenuated viruses, reassortant viruses, vaccine strains, as well as recombinant viruses and viral vectors derived thereof. Examples of viruses that may be used include, but are not limited to, poxviruses, orthomyxoviruses, paramyxoviruses, herpes viruses, hepadnaviruses, adenoviruses, parvoviruses, reoviruses, circoviruses, coronaviruses, flaviviruses, togaviruses, bimavriruses and retroviruses.

In some embodiments, the yield or virus and/or vaccine produced by the enriched population after infection is equal to or greater than a yield produced by the equivalent anchorage-dependent connective tissue cells. In some embodiments, the yield is greater than an equal number of equivalent anchorage-dependent connective tissue cells. In some embodiments, the yield is greater than the virus and/or vaccine produced by equivalent anchorage-dependent connective tissue cells in the same volume container. In some embodiments, the yield is at least 5, 10, 15, 20, 25, 30, 35, 40, 45, 50, 55, 60, 65, 70, 75, 80, 85, 90, 95 or 100% greater. Each possibility represents a separate embodiment of the invention.

In some embodiments, the enriched population is in medium. In some embodiments, the medium is serum-free medium. In some embodiments, the medium is chemically defined medium. In some embodiments, the enriched population is lyophilized. In some embodiments, the enriched population is in vitro. In some embodiments, the enriched population is ex vivo.

As used herein, the term “chemically defined medium” refers to growth medium suitable for in vitro culture of cells, in which all of the chemical components of the medium are known. Chemically defined media are well known in the art and any such media may be used, including those described herein, and for non-limiting example UltraCULTURE™ medium (Lonza), XerumFree™ medium (TNC Bio) and BIO-MPM-1 SFM (Biological Industries)

By another aspect, there is provided a composition comprising an enriched population of the invention and a liquid medium.

In some embodiments, the liquid medium is in vitro cellular growth medium. In some embodiments, the liquid medium is a suspension cell growth medium. In some embodiments, the liquid medium is adherent cell growth medium. In some embodiments, the liquid medium is chemically defined medium. In some embodiments, the medium is serum-free medium. In some embodiments, the liquid medium is freezing solution. In some embodiments, the composition is formulated to be thawed and resuspended in growth medium. In some embodiments, the freezing solution comprises DMSO. In some embodiments, the freezing solution comprises fetal bovine serum. In some embodiments, the liquid medium is a pharmaceutically acceptable solution. In some embodiments, the pharmaceutically acceptable solution comprises a pharmaceutically acceptable carrier, excipient or adjuvant. In some embodiments, the liquid medium comprises an acid. In some embodiments, the acid is ascorbic acid. In some embodiments, the acid is pluronic acid.

As used herein, a “liquid in vitro growth medium” refers to a liquid containing the nutrient sufficient for in vitro growth of cells. In some embodiments, the medium is tissue culture medium. In vitro growth media and tissue culture media are well known in the art and may be tailored to the particular cells being grown. Any known medium may be used. In some embodiments, the medium contains serum. In some embodiments, the medium is serum-free. In some embodiments, the medium is chemically defined. In some embodiments, the medium is devoid of viral particles, and/or retroviral particles. In some embodiments, the medium is suspension-cell medium. In some embodiments, the medium comprises DMEM basal medium. In some embodiments, the medium comprises DMEM/F12 basal medium. In some embodiments, the medium comprises UltraCULTURE medium. In some embodiments, the medium comprises an antibiotic. In some embodiments, the medium is devoid of antibiotics. In some embodiments, the medium is supplemented with a surfactant. In some embodiments, the surfactant is a non-ionic surfactant. In some embodiments, the surfactant comprises pluronic acid. In some embodiments, the surfactant is pluronic F68. In some embodiments, the medium is supplemented with pluronic acid. In some embodiments, the medium is supplemented with pluronic F68. In some embodiments, the medium is supplemented with L-glutamine and/or a derivative thereof. In some embodiments, the medium is supplemented with GlutaMAX. In some embodiments, the medium is DMEM with 10% FBS, GlutaMAX and 0.01% pluronic F68. In some embodiments, the medium is DMEM with 15% FBS, GlutaMAX and 0.01% pluronic F68. In some embodiments, the medium is DMEM/F12 with 15% FBS, GlutaMAX and 0.01% pluronic F68. In some embodiments, the medium is UltraCULTURE, GlutaMAX with 0.01% pluronic F68. In some embodiments the medium is based on CHO cell medium, Examples of CHO medium, include, but are not limited to PowerCHO, PeproGrow, and EX-CELL.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the composition is devoid of other cells than the cells of the invention. In some embodiments, the composition is devoid of support cells that adhere to the cells of the invention. In some embodiments, the composition is devoid of genetically modified additives. In some embodiments, the composition is devoid of human components.

In some embodiments, the cells of the enriched population are single cells in the medium. In some embodiments, at least 70%, 75%, 80%, 85%, 90%, 95%, 97%, 99% or 100% of the cells in the medium are growing as single cells. Each possibility represents a separate embodiment of the invention. In some embodiments, the between 70-100% of the cells are growing as single cells. In some embodiments, the medium contains serum and at least 90% of the cells are growing as single cells. In some embodiments, the medium contains serum and 90-100% of cells are growing as single cells. In some embodiments, the medium is serum-free and at least 70% of cells are growing as single cells. In some embodiments, the medium is serum-free and at least 80% of cells are growing as single cells. In some embodiments, the medium is serum-free and between 70-90% of cells are growing as single cells. In some embodiments, the medium is serum-free and at least 90% of cells are growing as single cells. In some embodiments, the medium is serum-free and greater than 5, 10, 15, 20, 25, 30, 35, 40, 45 or 50% of cells are growing as single cells. Each possibility represents a separate embodiment of the invention.

In some embodiments, at least 50, 55, 60, 65, 70, 75, 80, 85, 90, 95, 97, 99 or 100% of the anchorage-independent connective tissue cells are not adhered to a surface. Each possibility represents a separate embodiment of the invention. In some embodiments, the surface is an artificial surface. In some embodiments, the surface is a surface of the container holding the medium. In some embodiments, the surface is another cell. In some embodiments, the surface is a microcarrier. In some embodiments, the composition is devoid of microcarriers. As used herein, the term “microcarrier” refers to support matrix or scaffold allowing for the growth of cells in a liquid culture. In some embodiments, the microcarrier is for growth of adherent cells in a non-adherent container. In some embodiments, the microcarrier is for growth in a bioreactor. In some embodiments, the non-adherent containing is a bioreactor. In some embodiments, a microcarrier is an artificial scaffold for adherent cells to adhered to. In some embodiments, the microcarrier is a microcarrier bead. As used herein, growth while adhered to a microcarrier is not anchorage-independent growth, as the cell is anchored to the microcarrier.

In some embodiments, the anchorage-independent connective tissue cells are at a density of at least 1, 2, 3, 4, 5, 6, 7, 8, 9, 10 million cells/mL of in vitro cellular growth medium. Each possibility represents a separate embodiment of the invention. In some embodiments, the anchorage-independent connective tissue cells are at a density of at least 5 million cells/mL. In some embodiments, the anchorage-independent connective tissue cells are at a density of more than 1, 2, 3, 4, 5, 6, 7, 8, 9, 10 million cells/mL of in vitro cellular growth medium. Each possibility represents a separate embodiment of the invention. In some embodiments, the anchorage-independent connective tissue cells are at a density of more than 5 million cells/mL. In some embodiments, the anchorage-independent connective tissue cells are at a density greater than can be achieved by growing the equivalent anchorage-dependent cells in the same volume. One of the particular advantages of the anchorage-independent cells is that they can be grown at a far greater density and in larger numbers in the same space as compared to equivalent anchorage-dependent cells. This allows for the production of great numbers of cells, greater quantities of virus/vaccine and greater amounts of cultured meat.

By another aspect, there is provided an artificial meat composition, comprising the enriched population of the invention, wherein the anchorage-independent connective tissue cells are differentiated to adipocytes, myocytes, chondrocytes, osteocytes or a combination thereof. In some embodiments, the artificial meat composition comprises adipocytes. In some embodiments, the artificial meat composition comprises at least 70%, 75%, 80%, 85%, 90%, 95%, 97%, 99% or 100% adipocytes, myocytes, chondrocytes, osteocytes or a combination thereof. Each possibility represents a separate embodiment of the invention.

By another aspect, there is provided a method of producing an anchorage-independent cell line, the method comprising:
a. growing aggregates of an anchorage-dependent cell line in vitro;
b. mechanically disrupting the aggregates into single cells; and
c. growing the single cell in a liquid culture for at least 4 passages;
   thereby producing the anchorage-independent cell line.

By another aspect, there is provided a method of decreasing the doubling time of an anchorage-dependent cell line, the method comprising:
a. growing aggregates of the anchorage-dependent cell line in vitro;
b. mechanically disrupting the aggregates into single cells; and
c. growing the single cell in a liquid culture for at least 4 passages;
   thereby decreasing the doubling time of the anchorage-independent cell line.

In some embodiments, the anchorage-independent cell line is an enriched population of the invention. In some embodiments, the anchorage-dependent cell line is a connective tissue cell line and the anchorage-independent cell line is a cell line of the same connective tissue. In some embodiments, the anchorage-dependent cell line is a fibroblast cell line and the anchorage-independent cell line is a fibroblast cell line. In some embodiments, the fibroblast cell line is DF-1 and the anchorage-independent cell line is an anchorage-independent DF-1 line. In some embodiments, the anchorage-dependent cell line is a commercially available cell line. In some embodiments, the anchorage-dependent cell line is derived from primary cells. In some embodiments, the primary cells are immortalized to produce the anchorage-dependent cell line.

In some embodiments, the growing aggregates is performed in a non-adherent dish. In some embodiments, the non-adherent dish is a petri dish. In some embodiments, the non-adherent dish is an Aggrewell dish. In some embodiments, the Aggrewell dish is a Aggrewell 800 dish. In some embodiments, the non-adherent dish is a hydrogel microstructure array. In some embodiments, the non-adherent dish is an InSphereo dish. In some embodiments, the non-adherent dish comprises at least 6, 12, 24, 48, 72, 96, 128, 256, 300, 400, 500, 600, 700, 800, 900 or 1000 wells. Each possibility represents a separate embodiment of the invention. In some embodiments, the dish comprises small wells such that only a single aggregate or spheroid can form. In some embodiments, each well is seeded with between 1000-10000, 1000-9000, 1000-8000, 1000-7000, 1000-6000, 1000-5000, 1000-4000, 1000-3000, 2000-10000, 2000-9000, 2000-8000, 2000-7000, 2000-6000, 2000-5000, 2000-4000, 2000-3000, 3000-10000, 3000-9000, 3000-8000, 3000-7000, 3000-6000, 3000-5000, or 3000-4000 cells. Each possibility represents a separate embodiment of the invention. In some embodiments, each well is seeded with between 3000-4000 cells. In some embodiments, aggregates are grown for at least 12, 18, 24, 36 or 48 hours before mechanical disruption. Each possibility represents a separate embodiment of the invention.

In some embodiments, the method further comprises before mechanical disruption moving the aggregates to a non-adherent dish pre-coated with a surfactant. In some embodiments, mechanic disruption comprises vigorous pipetting. In some embodiments, the mechanic disruption is repeated over an extended period of time. In some embodiments, the extended period of time is at least 1, 2, 3, 4, 5, 6, or 7 days. Each possibility represents a separate embodiment of the invention.

In some embodiments, the growing single cells is performed in a shaker or spinner flask. In some embodiments, the growing single cells is performed in a shaker flask. In some embodiments, the growing single cells is performed in a spinner flask. In some embodiments, the growing single cells comprises growing first at high density with little or no shaking followed by shaking at a higher speed. In some embodiments, little or no shaking is at most 40, 35, 30, 25, 20, 15, 10, 5, 3, 2, 1 or 0 revolutions per minute (RPM). Each possibility represents a separate embodiment of the invention. In some embodiments, little or no shaking is 40 revolutions per minute (RPM) or less. In some embodiments, the little of no shaking is for at most 6, 12, 18 or 24 hours. Each possibility represents a separate embodiment of the invention. In some embodiments, the little of no shaking is for at most 1, 2, 3, 4, or 5 passages. In some embodiments, the little of no shaking is for at most 1 passage. In some embodiments, the little or no shaking is overnight.

In some embodiments, higher speed shaking is at least 60, 80, 100, 120, 140 or 160 RPM. Each possibility represents a separate embodiment of the invention. In some embodiments, higher speed shaking is between 60-160, 60-140, 60-120, 60-100, 60-90, 60-80, 80-160, 80-140, 80-120, 80-100, or 80-90 RPM. Each possibility represents a separate embodiment of the invention. In some embodiments, the higher speed shaking is for at least 2, 3, 4, 5, 7, or 10 passages. Each possibility represents a separate embodiment of the invention.

In some embodiments, shaking is performed at an initial speed and then increased to a higher speed. In some embodiments, the initial speed is about 40, 50, 60, 70, 80, 90, or 100 RPM. Each possibility represents a separate embodiment of the invention. In some embodiments, the initial speed is at most 40, 50, 60, 70, 80, 90, or 100 RPM. Each possibility represents a separate embodiment of the invention. In some embodiments, the initial speed is at least 40, 50, 60, 70, 80, 90, or 100 RPM. Each possibility represents a separate embodiment of the invention. In some embodiments, the higher speed is about 60, 70, 80, 90, 100, 110, 120, 130, 140, 150, 160, 170, 180, 190, or 200 RPM. Each possibility represents a separate embodiment of the invention. In some embodiments, the high speed is at most 60, 70, 80, 90, 100, 110, 120, 130, 140, 150, 160, 170, 180, 190, or 200 RPM. Each possibility represents a separate embodiment of the invention. In some embodiments, the high speed is at least 60, 70, 80, 90, 100, 110, 120, 130, 140, 150, 160, 170, 180, 190, or 200 RPM. Each possibility represents a separate embodiment of the invention. In some embodiments, the initial speed is 80 RPM and the higher speed is 100 RPM.

In some embodiments, the increase occurs after passage 1, 2, 3, 4 or 5. Each possibility represents a separate embodiment of the invention. In some embodiments, the increase occurs after passage 3. In some embodiments, the increase occurs before passage 2, 3, 4, 5 or 6. Each possibility represents a separate embodiment of the invention. In some embodiments, the increase occurs between passages 1 and 6, 1 and 5, 1 and 4, 1 and 3, 2 and 6, 2 and 5, 2 and 4, 2 and 3, 3 and 6, 3 and 5, or 3 and 4. Each possibility represents a separate embodiment of the invention.

In some embodiments, the method further comprises transfer to a bioreactor. In some embodiments, the method further comprises culturing for 2, 5, 7, 10, 15, 20, 25, 30, 34 or 35 passages. Each possibility represents a separate embodiment of the invention.

In some embodiments, the decreasing is at least a 10%, 15%, 20%, 25%, 30%, 35%, 40%, 45%, 50%, 55%, 60%, 65%, 70%, 75%, or 80% decrease in doubling time. Each possibility represents a separate embodiment of the invention. In some embodiments, the decreasing is at least a decrease of 1, 2, 3, 4, 5, 6, 7, 8, 9, 10, 12, 15, 17, 20 hours. Each possibility represents a separate embodiment of the invention.

In some embodiments, cells are diluted to a desired concentration. In some embodiments, the cells are diluted to at or below a desired concentration. In some embodiments, the desired concentration is about 600,000 cells/mL. In some embodiments, the desired concentration is about 400,000, 500,000, 600,000, 700,000 or 800,000 cells/mL. Each possibility represents a separate embodiment of the invention. In some embodiments, the desired concentration is between 400,000 and 800,000, 400,000 and 700,000, 400,00 and 600,000, 500,000 and 800,000, 500,000 and 700,000, 500,000 and 600,000, 600,000 and 800,000, 600,000, 700,00 cells/mL. Each possibility represents a separate embodiment of the invention.

In some embodiments, cells are diluted when they reach an undesired concentration. In some embodiments, cells are diluted when they reach or are above an undesired concentration. In some embodiments, the undesired concentration is about 1,200,000 cells. In some embodiments, the undesired concentration is about 1,000,000 cells. In some embodiments, the undesired concentration is about 800,000, 900,000, 1,000,000, 1,100,000, 1,200,000, 1,300,000, 1,400,000, or 1,500,000 cells/mL. Each possibility represents a separate embodiment of the invention. In some embodiments, the undesired concentration is between 800,000 and 1,500,000, 800,000 and 1,300,000, 800,000 and 1,200,000, 800,000 and 1,000,000, 900,000 and 1,500,000, 900,000 and 1,300,000, 900,000 and 1,200,000, 900,000 and 1,000,000, 1,000,000 and 1,500,000, 1,000,000 and 1,300,000, 1,000,000 and 1,200,000, or 1,000,000 and 1,100,000. Each possibility represents a separate embodiment of the invention.

By another aspect, there is provided a method of producing an anti-viral vaccine, the method comprising infecting the enriched population of the invention with said virus, growing said population for a time sufficient for viral particles to be produced and harvesting the viral particles, thereby producing a viral vaccine.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Materials

DMEM, DMEM/F12 basal medium and Polaxamer 188 solution F-68 (Pluronic®) were purchased from Sigma-Aldrich. L-Analyl L-Glutamine (GlutaMAX), heat-inactivated fetal bovine serum (FBS), penicillin-streptomycin, and trypsin EDTA were purchased from Biological Industries. TypLE™ enzyme was purchased from Fisher Scientific. Aggrewell 800 was bought from STEMCELL Technologies. TriForest shaker flasks were purchased from TriForest Labware, while T75 cell culture flasks were purchased from Greiner Bio-one.

### Cell Source and Media

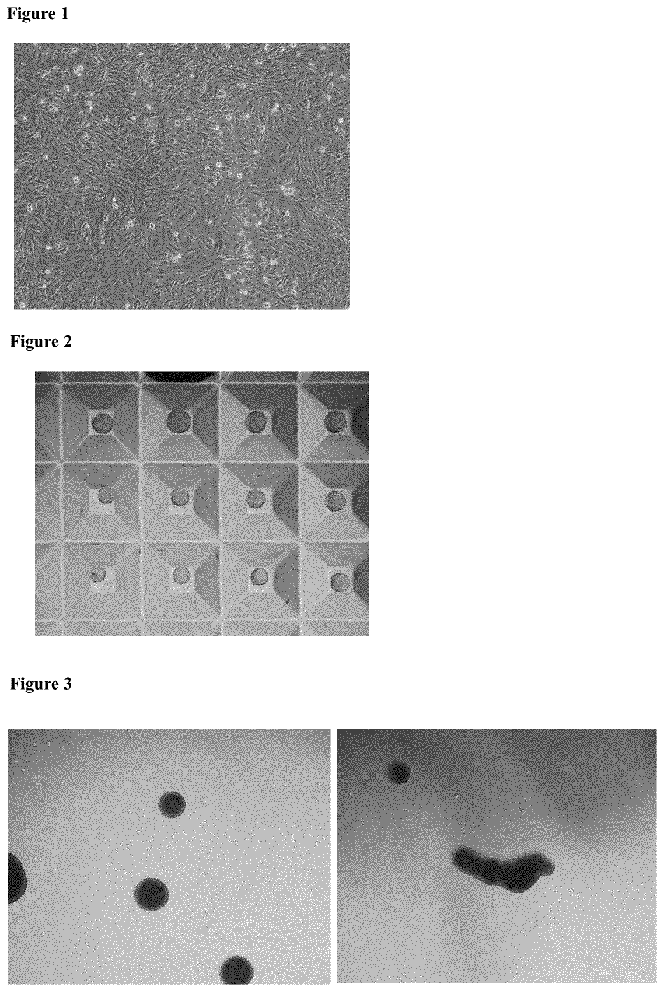

The UMNSAH/DF-1 (ATCC: CRL-12203) was purchased from ATCC and grown at 39° C., in a humidified tissue culture incubator under 5% CO₂ (FIG. 1).

Chicken fibroblasts were isolated from specific pathogen free (SPF) eggs on day 11, and spontaneously immortalized in culture. Culture medium was DMEM supplemented with 10% FBS, L-analyl-L-Glutamin and Penicillin Streptomycin.

Fetal bovine fibroblasts were isolated from specific pathogen free (SPF) fetuses, and spontaneously immortalized in culture. Culture medium was DMEM supplemented with 10% FBS, L-analyl-L-Glutamin and Penicillin Streptomycin. Adult bovine fibroblasts were isolated from dermis sections, obtained from Kosher slaughtered beef carcasses under veterinary supervision. Cells were obtained by outgrowth and spontaneously immortalized in culture. Culture medium was DMEM supplemented with 10% FBS, L-analyl-L-Glutamin and Penicillin Streptomycin.

### Example 1: Spheroid-Based Adaptation to Suspension Culture

1.16 million cells of the DF-1 chicken fibroblast cell line were seeded in Aggrewell 800 plate (3867 cells/microwell). Following 24-hour incubation, half of the medium in the well was replaced. The next day, the spheroids that formed within the aggrewell (FIG. 2) were mechanically detached and transferred to a non-adherent 10 cm petri dish pre-coated with Pluronic® F-68 in culture medium supplemented with 0.01% F-68. Cell aggregates were mechanically disrupted by vigorous pipetting during 4 consecutive days of culture (FIG. 3). On Day 7 of culture, the spheroids were transferred into a shaker incubator, in 3 ml of culture medium supplemented with 0.01% F-68 and shaken at a speed of 80, 100 or 140 RPM for 3 days. At the end of the adaptation process, trypan blue exclusion assay showed a cell density of 400,000 to 800,000 cells/mL with viability of 79%.

### Example 2: Direct Shaker Flask Adaptation to Suspension Culture

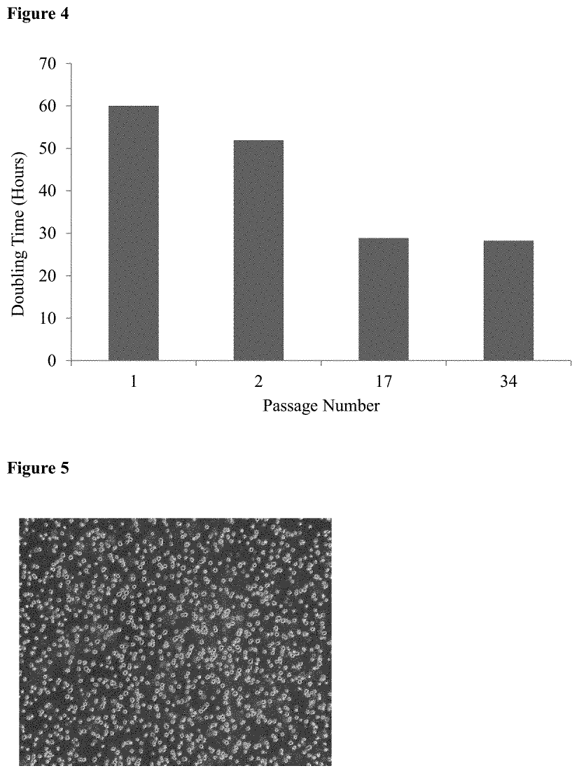

16 to 40 million cells from the adaptation process were seeded directly in 250 mL shaker flasks containing 80 ml of culture medium supplemented with 0.01% F-68 to a final density of 200,000 to 500,000 cells/mL. Cell were allowed to settle and aggregate overnight, then transferred to shaker incubator at 80 RPM. Cell growth was monitored and recorded over time. At each passage, cells were enzymatically digested and counted before reseeding at 200,000 cells/mL. Shaker speed was increase from 80 RPM to 100 RPM at passage 3. Doubling time decreased from 60 to 52 hours over the first two passages, stabilizing at 20-28 hours within 5 to 7 passages (FIG. 4). Aggregates became less and less frequent with each passage, reaching 5-10% of the culture by passage 34 (FIG. 5). Eventually at low concentration of cells the percentage of aggregates becomes even less than 5%.

### Example 3: Direct Spinner Flask Adaptation to Suspension Culture

Similarly, 16 million cells from the adaptation process were seeded directly in 250 mL Corning glass spinner flask containing 80 ml of culture medium supplemented with 0.01% F-68 to a final density of 200,000 cells/mL. Cell were allowed to settle and aggregate overnight, then spun at 60 to 90 RPM. Cell growth was monitored and recorded over time. At each passage, cells were enzymatically digested and counted before reseeding at 200,000 cells/mL. Doubling time and culture behavior was equivalent to that observed in shaker flasks. First generation doubling time was 62 hours.

### Example 4: Adaptation to Serum-Free Culture Medium

16 million cells from the adaptation process were split into 250 mL shaker flasks containing 80 ml of culture medium supplemented with 0.01% F-68 to a final density of 200,000 cells/mL. Cells reached a density of 1.2 million cells/mL by Day 3 of culture. Culture was then diluted to 600,000 cells/mL by addition of 80 mL UltraCULTURE medium to each flask. The next day the cells reached a density of 1 million cells/mL and were diluted again in serum-free medium to an FBS concentration of 2.5%. Cells reached 1.2 million cell/mL within 24 hours and were harvested following 5 min centrifugation at 300 g. The resulting cell pellet was finally re-suspended in UltraCULTURE serum-free medium and the cells were cultured in suspension in the absence of serum at passages 10, 26 and 34.

### Example 5: Growth in a Scalable Stirred Bioreactor

400 million cells at passage 34 (serum-free) were seeded in a 2-liter glass bioreactor (Sartorius) controlled by BIOSTAT A unit to a final concentration of 400,000 cells/mL in UltraCULTURE. Cultures were maintained at 275 RPM, at set-point pH of 7.1 and 60% oxygen saturation. Cells expanded with doubling time of 20 hours, and viability around 95%. A maximum concentration of 30 million cells/mL was possible in a Fed-Batch reactor and 250 million cells/mL in a perfusion reactor.

### Example 6: Anchorage-Independent Growth of Immortalized Primary Chicken and Bovine Fibroblasts

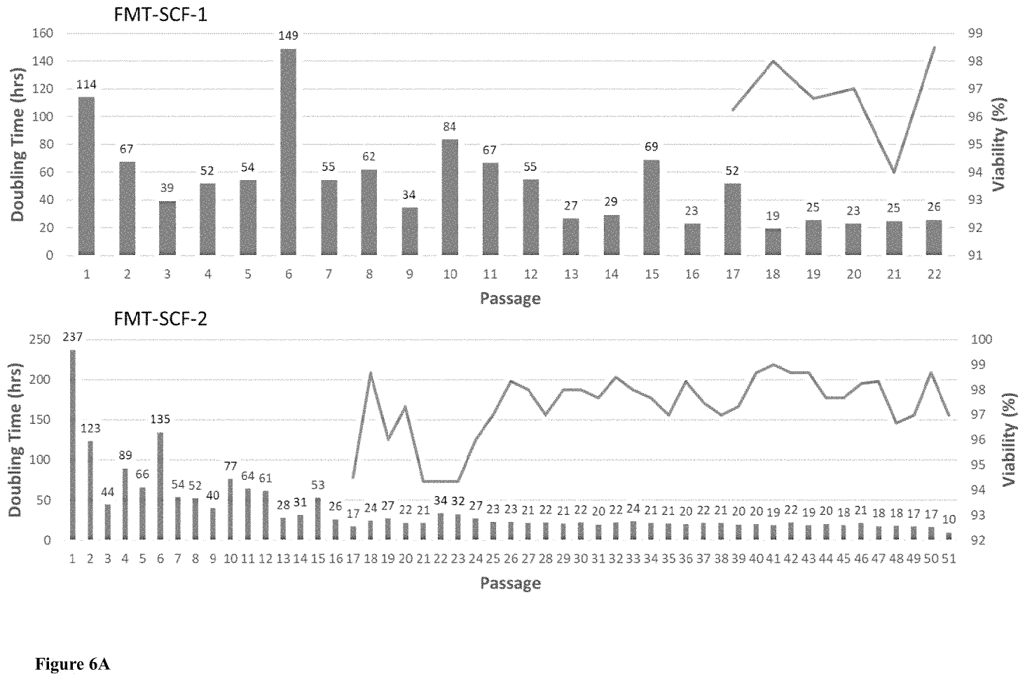

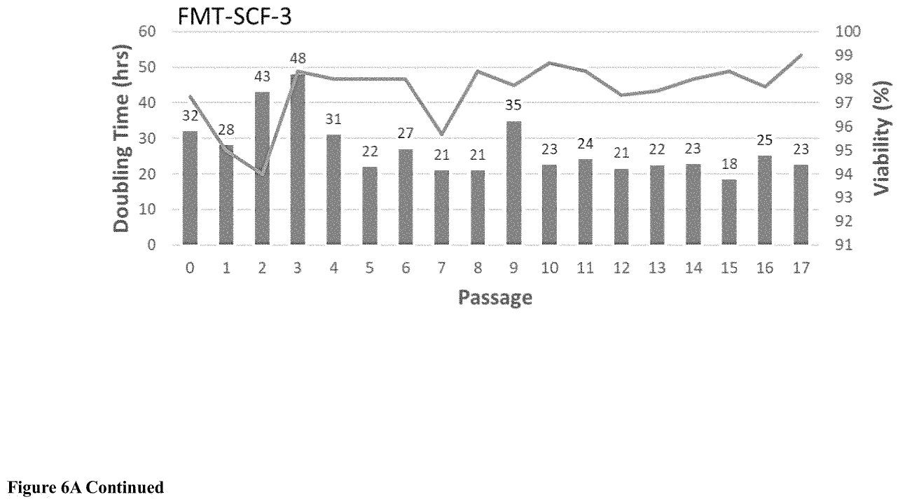

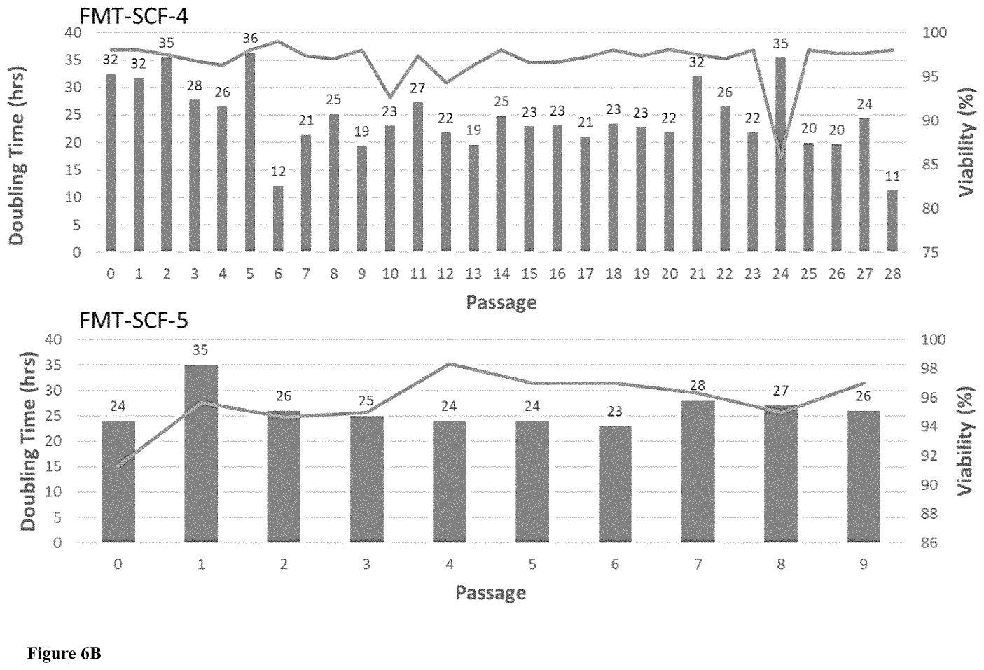

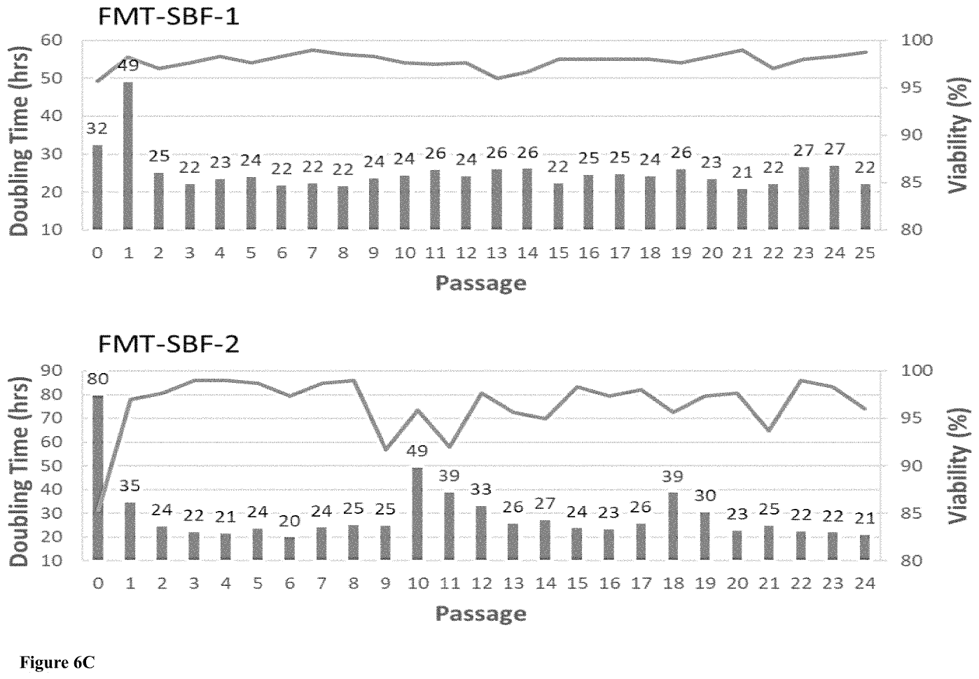

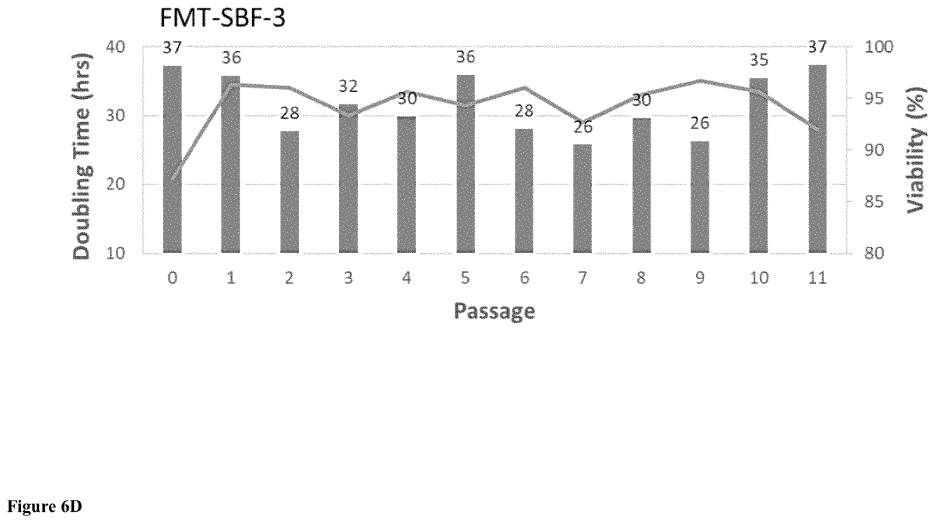

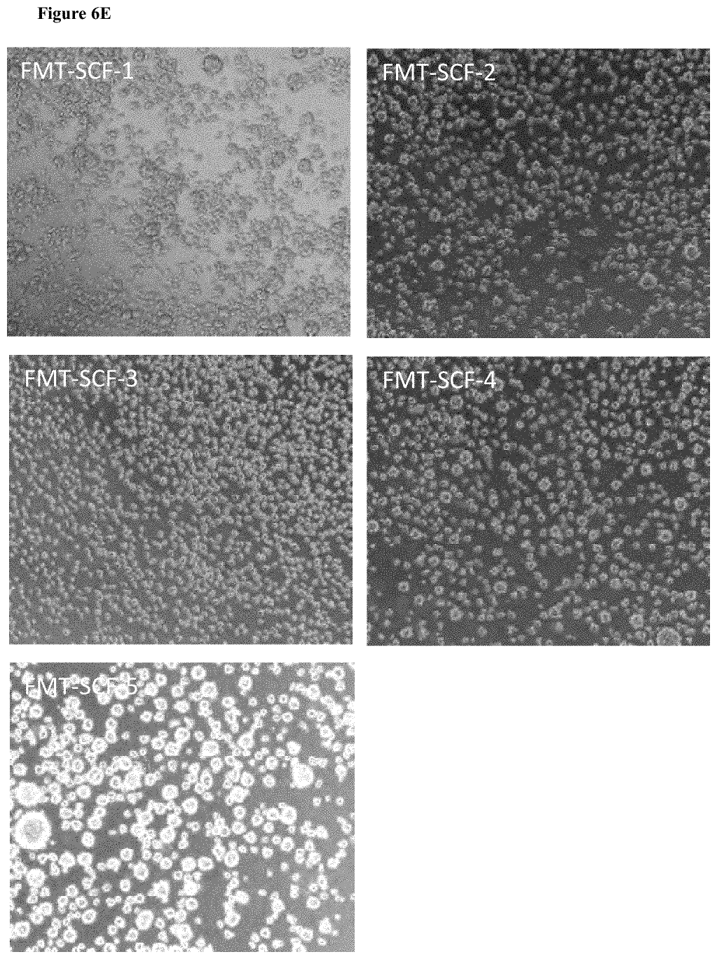

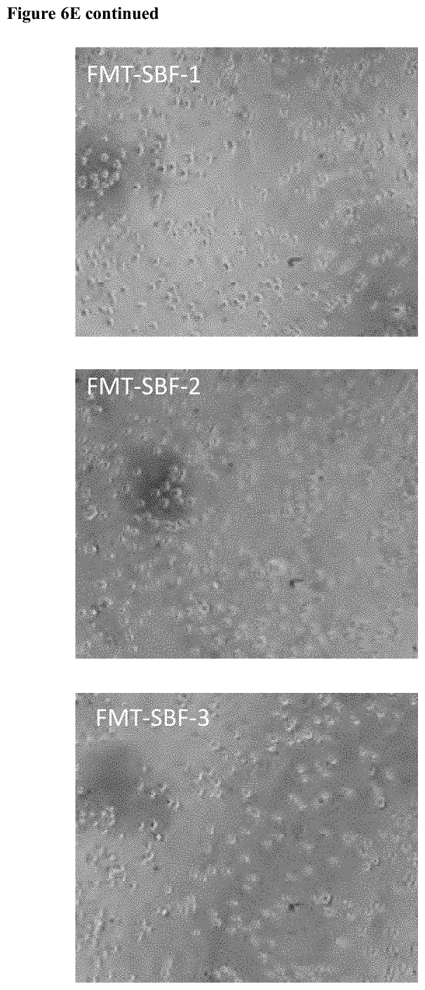

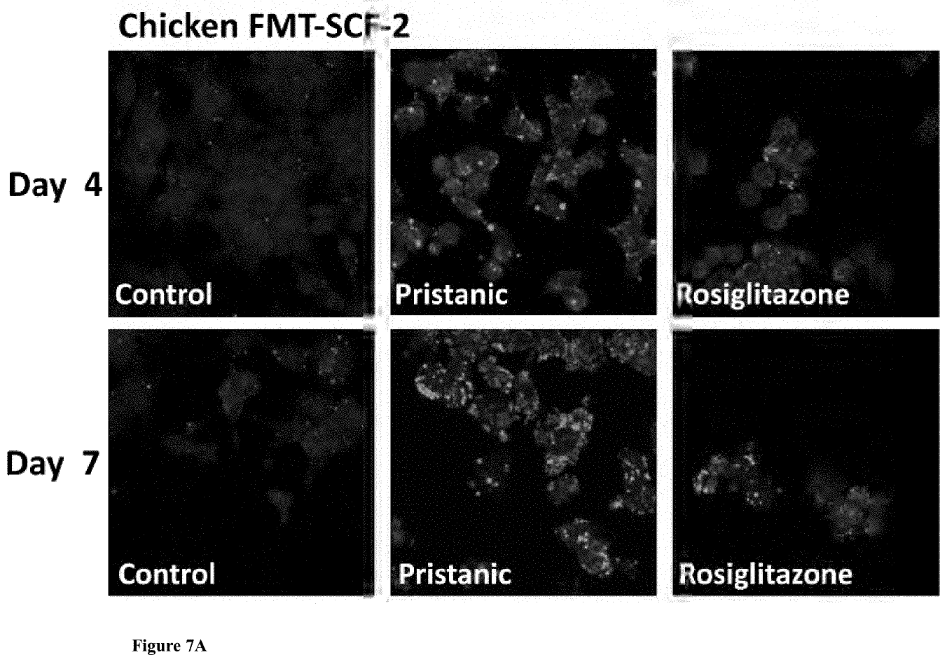

In addition to commercially available cell lines, different lines of chicken and bovine fibroblasts were generated from primary cells that were immortalized. FMT-SCF-1 and FMT-SCF-2 were derived from spontaneously immortalized fetal chicken fibroblasts of Broiler Ross308 chicken embryos (FIG. 6A, E), and lines FMT-SCF-3, SCF-4, and SCF-5 were derived from Israeli Baladi chicken embryonic fibroblasts (FIG. 6B, E). FMT-SBF-1 and FMT-SBF-2 were derived from spontaneously immortalized fetal bovine fibroblasts of Black Angus cattle (FIG. 6C, E), and line FMT-SBF-3 was derived from spontaneously immortalized dermal fibroblasts of Belgium Blue cattle (FIG. 6D-E).

Both avian and bovine fibroblasts were converted from anchorage-dependent to anchorage-independent as described for the DF-1 cells. Specifically, the shaker flask method was employed with shaking at 100 RPM in a humidified incubator at 5% CO₂. Chicken cells were cultured at 39° C. and bovine cells at 37° C. Cells were passaged every 3 days and reseeded at 0.3 million cells/ml. The resultant cell lines were 100% anchorage-independent, with no cells observed adhering to the container. The same had been observed for the DF-1 cells and thus these cells lines are truly anchorage-independent.

FMT-SCF-1 and FMT-SCF-2 reached a stable doubling time of between 18 and 25 hours after about 16-18 passages (FIG. 6A). At earlier passages, doubling times greater than 100 hours were observed, with most doublings taking at least 40 hours. Viability was consistently above 94% and generally above 97%.

FMT-SCF-3, FMT-SCF-4, and FMT-SCF-5 showed greater variability, but on average reached stable doubling times of between 21 and 26 hours (FIG. 6B). For FMT-SCF-3 a drop in doubling time from over 40 hours was already seen by passage 5, which was also observed for FMT-SCF-4 by passage 6. FMT-SCF-5 had a decreased doubling time from the initial time point, with only one measurement above 30 hours (at passage 1). Viability was again consistently above 94%.

FMT-SBF-1 and FMT-SBF-2 showed high doubling times in the first few passages that generally stabilized to between 22 and 27 hours (FIG. 6C). Viability was once again consistently above 94% and generally above 97%. FMT-SBF-1 was particularly stably, while FMT-SBF-2 showed a few passages with longer doubling times. FMT-SBF-3 showed a lower doubling time in the high thirties even at the initial passage. Doubling time did decrease to an average of about 30 hours, although with some variability ranging from 26-36 hours (FIG. 6D). Viability was also good and consistently above 90%, with an average of about 95%.

All of the anchorage-independent cell lines derived from primary cells showed greater than 90% single cells in culture (FIG. 6E). Only very small clumps of a few cells were observed for any of the cell lines when they were grown at high density. At low density the cell lines grew as over 97% single cells.

Anchorage-independent cells derived from primary cells are also derivable using the spinner flask method. They can also be grown in serum-free media and can be scaled up for a stirred bioreactor.

### Example 7: Generation of Anchorage-Independent Adipocytes and Cultured Meat

Chicken and bovine anchorage-independent fibroblasts were differentiated into anchorage-independent adipocytes by standard differentiation protocols. FMT-SCF-2 (chicken non-adherent fibroblasts) and FMT-SBF-1 (bovine non-adherent fibroblast) were grown in adipogenesis medium containing 200 μM oleic acid together with a PPARgamma agonists. A synthetic inhibitor (Rosiglitazone) and a natural inhibitor (Pristanic acid) were both tested.

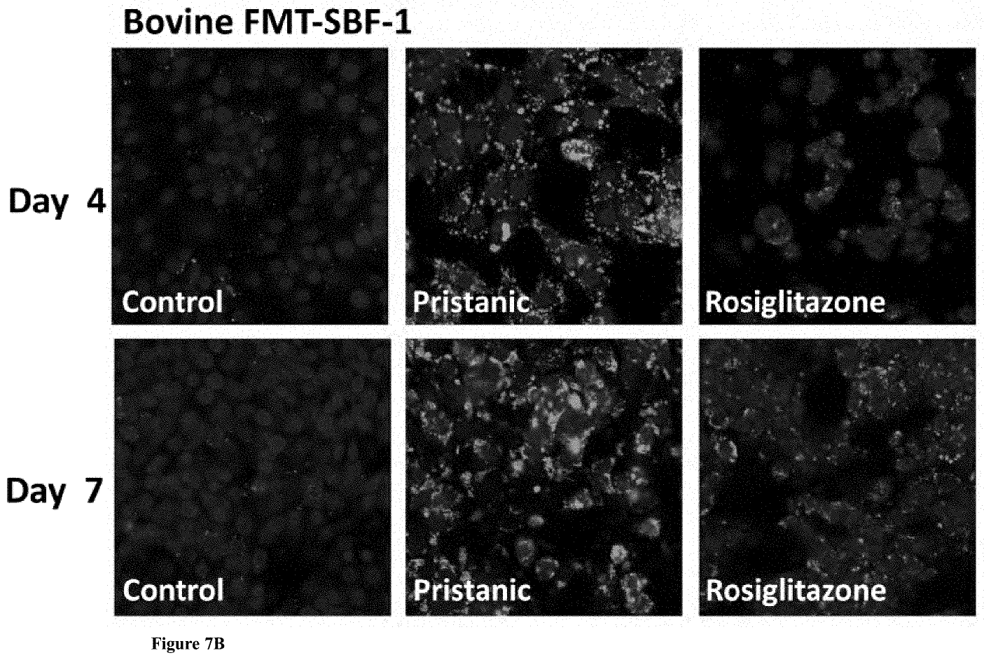

To determine that differentiation to adipocytes had occurred the cells were assayed for lipid production. On days 4 and 7 cells were harvested, reseeded in a black 96 well plate, incubated for 2 hours and then fixed in 4% PFA. The fixed cells were stained with LipidTOX, which stains neutral lipid droplets green. The cell nuclei were counter stained blue with Hoechst. At day 4, cells were already staining positive for lipid droplets indicating the presence of adipocytes; the staining was increased at day 7 (FIG. 7A-B). Lipid production was seen in both chicken cells (FIG. 7A) and bovine cells (FIG. 7B), and with both the synthetic and natural inhibitors.

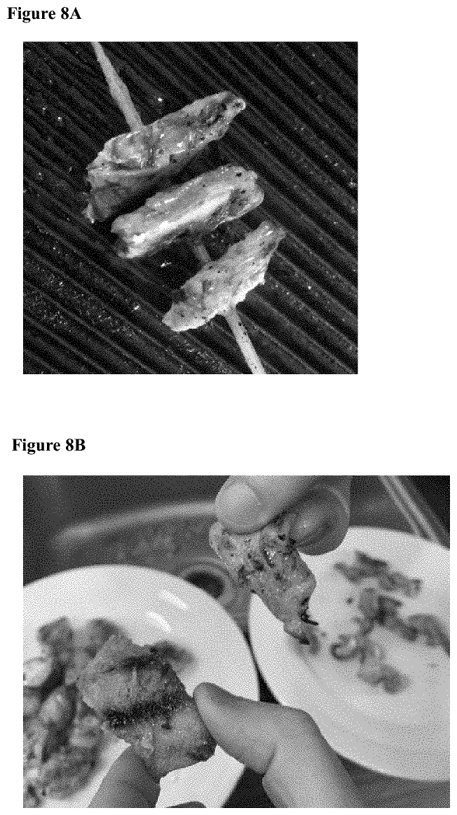

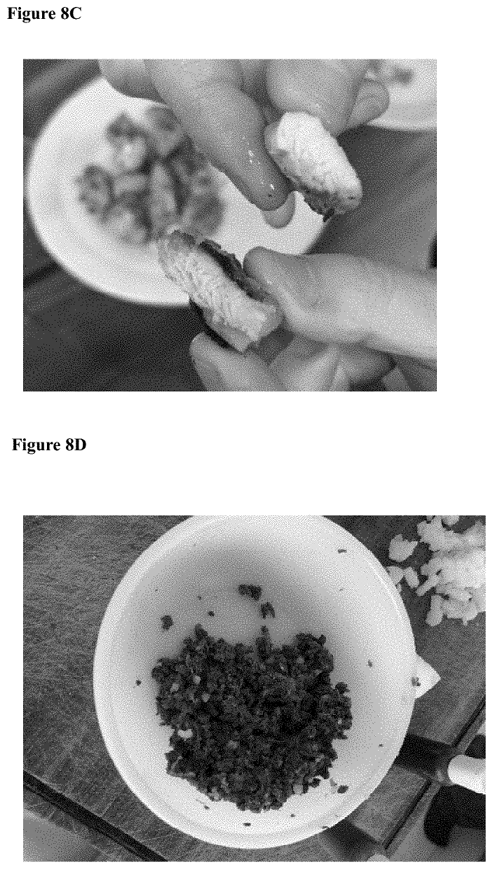

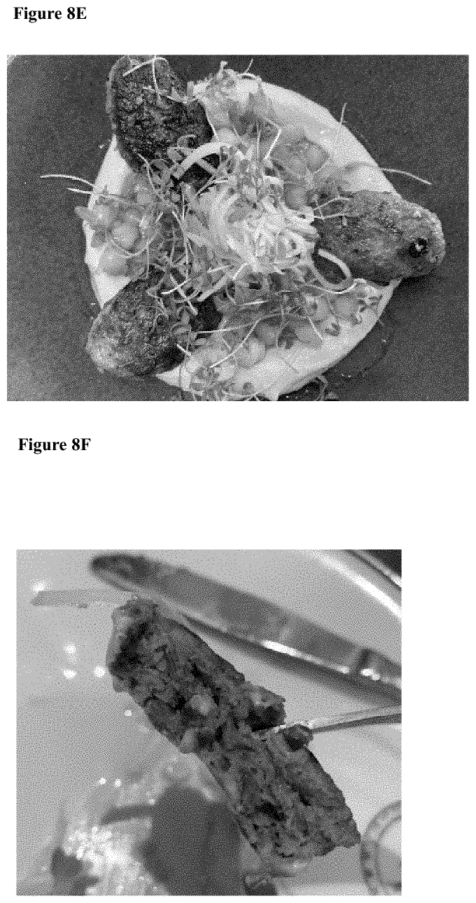

The anchorage-independent adipocytes were used to make cultured meat according to a standard protocol. First, chicken adipocytes were combined with high moisture extrusion of soy protein. A ratio of 20% adipocytes and 80% soy protein by weight was used. The fat cells could be directly mixed with the soy protein or was coated by the soy protein to produce cultured chicken nuggets (FIG. 8A). The final product contained about 11% total fat, less than 1% of which was saturated fat; 1% carbohydrates, of which less than 1% was sugars; and about 19% protein. The cultured chicken compared favorably to farm grown chicken in external (FIG. 8B) and internal (FIG. 8C) look, texture and taste.

Cultured beef was also produced. Bovine adipocytes were combined with textured wheat proteins to produce a mixture comparable to ground beef (FIG. 8D). Similarly, the adipocytes were mixed with textured soy protein to produce beef kabobs (FIG. 8E-F). A ratio of 30% adipocytes and 70% textured proteins, by weight, was used. The final product contained about 18% fat, less than 1% of which was saturated fat, about 7% carbohydrates, of which less than 1% was sugars, and about 17% protein. The cultured beef compared favorably to farm grown beef in both external (FIG. 8E) and internal (FIG. 8F) look, texture and taste.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. An enriched population of connective tissue cells, wherein at least 70% of said connective tissue cells are capable of anchorage-independent growth.

2. (canceled)

3. (canceled)

4. The enriched population of claim 1, wherein at least 20% of said anchorage independent connective tissue cells are actively proliferating.

5. The enriched population of claim 1, wherein said anchorage-independent connective tissue cells are capable of anchorage-independent growth for at least 4 cellular divisions.

6. (canceled)

7. The enriched population of claim 1, wherein said connective tissue cells are fibroblasts.

8. The enriched population of claim 1, wherein said connective tissue cells are a cell type that can naturally be differentiated from a fibroblast and is selected from the group consisting of: a chondrocyte, an adipocyte, an osteoblast, an osteocyte, a myofibroblast, a myoblast and a myocyte.

9. The enriched population of claim 1, wherein said anchorage-independent connective tissue cells comprise an intact plasma membrane.

10. The enriched population of claim 1, wherein said enriched population comprises a doubling time of 50 hours or less.

11. (canceled)

12. The enriched population of claim 1, wherein said anchorage-independent connective tissue cells grow in liquid culture as at least 85% single cells.

13. (canceled)

14. (canceled)

15. The enriched population of claim 1, wherein said connective tissue cells are capable of producing cultured meat.

16. The enriched population of claim 1, wherein a yield of virus produced by said enriched population after infection is equal to or greater than a yield produced by an equal number of anchorage-dependent connective tissue cells after infection.

17. (canceled)

18. (canceled)

19. A composition comprising the enriched population of claim 1, the composition further comprising a liquid in vitro cellular growth medium, wherein at least 70% of said anchorage-independent connective tissue cells are not adhered to a surface, and wherein said in vitro cellular growth medium is devoid of serum.

20. (canceled)

21. (canceled)

22. (canceled)

23. (canceled)

24. The composition of claim 19, further comprising a matrix wherein said matrix is a vegetable-derived matrix, wherein said anchorage-independent connective tissue cells are differentiated into adipocytes and wherein said composition is cultured meat.

25. The composition of claim 19, further comprising a matrix wherein said matrix is selected from a collagen matrix, a dermal matrix and a substitute dermal matrix and wherein said composition is leather.

26. A method of producing an anchorage-independent cell line, the method comprising:
a. growing aggregates of an anchorage-dependent cell line in vitro;
b. mechanically disrupting said aggregates into single cells in liquid; and
c. growing said single cells in a liquid culture for at least 4 generations;
thereby producing said anchorage-independent cell line.

27. A method for decreasing the doubling time of an anchorage-dependent cell line, the method comprising:
a. growing aggregates of said anchorage-dependent cell line in vitro;
b. mechanically disrupting said aggregates into single cells in liquid; and
c. growing said single cells in a liquid culture for at least 4 generations;
thereby increasing the doubling time of said anchorage-dependent cell line.

28. (canceled)

29. The method of claim 26, wherein said growing single cells is performed in shaker or spinner flasks.

30. The method of claim 29, wherein said growing in shaker or spinner flasks comprises at most one passage at spin speeds below 40 RPM, followed by at least 3 passages at spin speeds of between 80 and 100 RPM.

31. The method of claim 26, wherein said anchorage-dependent cell line is a fibroblast cell line, and wherein said anchorage-independent cell line is a fibroblast cell line.

32. The method of claim 31, wherein said anchorage-independent cell line is the enriched population of claim 1.

33. (canceled)
